(19) United States
(12) Patent Application Publication
(10) Pub. No.: US 2022/0365605 A1
(43) Pub. Date: Nov. 17, 2022

(54) SYSTEM AND METHOD FOR LEARNING OR RE-LEARNING A GESTURE

(71) Applicant: Vector Labs, Prémanon (FR)

(72) Inventors: GREGOIRE Lucie, Rieux-Volvestre (FR); LAGIER Paul, Prémanon (FR)

(73) Assignee: Vector Labs, Prémanon (FR)

(21) Appl. No.: 17/763,188

(22) PCT Filed: Sep. 22, 2020
(86) PCT No.: PCT/FR2020/051653

§ 371 Date: Mar. 23, 2022

(30) Foreign Application Priority Data
Sep. 23, 2019 (FR) FR1910488

(51) Int. Cl. G06F 3/01; G10L 15/26

(52) U.S. Cl. CPC G06F 3/017; G10L 15/26

(57) ABSTRACT

The invention relates to a system and a method for learning a gesture by a human learner (5), comprising the following steps: equipping said learner (5) with a plurality of motion sensors (6, 7) on a plurality of members that are predetermined in accordance with said gesture to be learned; acquiring biomechanical data provided by said plurality of sensors during a gesture performed by the learner; analyzing said acquired biomechanical data and determining a theoretical correction of the gesture by comparing said biomechanical data of the learner with biomechanical data corresponding to a target gesture; customizing the theoretical correction into a specific correction on the basis of behavior models of the learner derived from a history of biomechanical data acquired for the learner; transmitting said specific correction to the learner; updating said specific correction on the basis of information representing the sensation perceived by the learner when performing the corrected gesture.

### TECHNICAL FIELD OF THE INVENTION

The invention relates to a system and a method for learning or relearning a gesture by a user, such as an athlete seeking to improve their sporting activity or a patient in a rehabilitation phase.

### TECHNOLOGICAL BACKGROUND

Learning a sporting activity or a rehabilitation phase following a trauma generally require a professional (sports coach, physical education teacher, trainer, physiotherapist or osteopath, etc.) who will guide the learning of the athlete or the patient in their rehabilitation program.

The role of this professional is to impart their understanding of the science of motion to the learner so that they can assimilate the required gesture and subsequently reproduce it in a satisfactory manner. For example, a tennis player is guided by their trainer to optimize how they hit the ball, i.e., to be able, in practice, to hit the ball when the racket of the player reaches its maximum speed. A long-distance runner is trained to be able to optimize their stride and reach a maximum speed, while minimizing the energy expenditure needed to maintain it over time. A golf player is guided to smooth out their swing and hit the ball with as much power as possible while controlling the ball's trajectory.

Throughout the remainder of the document, the term “learner” denotes both an athlete seeking to improve their sporting activity and a patient in a rehabilitation phase, and the term “learning” denotes both learning as such (i.e., achieving a specific gesture for the first time) and relearning a movement, the mastery of which has been forgotten or disrupted as a result of a trauma of any kind.

The term “gesture” denotes the various movements of the various parts of the body (arms, legs, hands or pelvis, etc.) that the learner must carry out in order to perform a predetermined gesture, such as hitting a tennis ball, a golf swing or a running stride, etc.

In general, the professional shows the learner how to perform the gesture and asks them to reproduce it. They then give the learner instructions, which result from the observation of the gesture performed by the learner in order to allow them to correct certain faults in performing the gesture. By way of an example, the correction instructions can involve asking the learner to turn their shoulders more toward the net in order to improve the hitting of a tennis ball, to move the pelvis forward during a golf swing or even to drive into the ground with the tip of the foot during a running session.

For several years hardware solutions have also existed that obviate the need for a professional when learning the gesture or relearning a gesture by a learner.

For example, WO2006081395 describes a system and a method for analyzing and learning a sporting gesture, in particular a golf swing, which does not involve an external human operator.

To this end, the system implements an optical system for acquiring images of the gesture performed by the learner, sensors worn by the learner and a computing unit configured to be able to analyze the data from the sensors and the images acquired by the camera.

The computing unit is configured to be able to break down the movement into a plurality of main biomechanical entities, to gather the data provided by the sensors worn by the learner, and to enhance this data with the images acquired by the optical system.

Furthermore, the system allows gesture correction instructions to be provided by automatically analyzing the gesture acquired by the camera, the measurements of the sensors and the target gesture which is prerecorded in the system.

The learner can then visualize their gesture, compare it to the optimal gesture which is prerecorded in the system, and perceive the modifications to be made to the gesture in order to achieve the target gesture.

Other substantially equivalent systems have been proposed by WO02/067187, U.S. Pat. Nos. 6,778,866, 9,248,361 and 9,851,374, etc., for a variety of physical activities (golf or baseball, etc.).

These different solutions are interesting but are not very effective in practice because each learner is specific and the targeted target gesture may not be adapted to the learner. For example, with reference to long-distance runners, each runner has their own technique, without one being considered, at first sight, to be more effective than another. However, it is important that each runner identifies the best technique which is adapted to their own morphology and to their own history.

### Aims of the Invention

The invention aims to provide a system and a method for learning a gesture by a learner that overcomes at least some of the disadvantages of the known systems and methods.

The invention particularly aims to provide a learning system and method which allow correction instructions to be provided before the volatile memory of the learner is dissipated.

The invention also aims to provide, in at least one embodiment, a learning system and method which do not require any human presence, with the exception of the learner.

The invention also aims to provide, in at least one embodiment, a learning system and method which can operate in real time.

The invention also aims to provide, in at least one embodiment, a learning system and method which can be embedded, without requiring any specific infrastructure.

The invention also aims to provide, in at least one embodiment, a learning system and method which can operate with a limited number of sensors.

The invention also aims to provide, in at least one embodiment, an interactive learning system and method.

### DISCLOSURE OF THE INVENTION

To this end, the invention relates to a method for learning a gesture by a human learner, the skeleton of said human learner being modeled by a plurality of members interconnected by links according to a parent-child inheritance relationship so that the movement of a parent member causes the movement of each child member connected to the parent member by a link, each link being associated with a range of motion and at least one degree of freedom in space.

The method according to the invention comprises at least the following steps:
equipping said learner with a plurality of motion sensors on a plurality of members that are predetermined in accordance with said gesture to be learned from among those that model said human skeleton;
acquiring biomechanical data provided by said plurality of sensors during a gesture performed by the learner;
analyzing said acquired biomechanical data and determining a theoretical correction instruction for the gesture on the basis of said modeled human skeleton and by comparing said biomechanical data of the learner with biomechanical data corresponding to a target gesture;
customizing said theoretical correction instruction into a specific correction instruction on the basis of predetermined adaptive parameters related to the learner and/or to the environment;
transmitting said specific correction instruction to the learner;
updating said specific correction instruction on the basis of information representing the sensation perceived by the learner when performing the corrected gesture.

A method according to the invention therefore allows a gesture to be learned or relearned by a human learner while taking into account the specific features of the learner and the sensation (of proprioceptive and/or environmental origin) felt by the learner when performing the gesture.

In other words, the invention allows both the gesture of the learner to be corrected, but also allows the specific features and the feelings (also called feedback) of the learner to be taken into account.

In particular, a method according to the invention allows a learner to be provided, in real time (or near real time), with relevant information relating to the nature of the performed gesture, any differences from the target gesture and the corrections needed to achieve this target gesture.

The invention allows the attractors of the learner to be taken into account, i.e., the specific features of each learner that cause some of their movements to irreversibly tend toward given positions, somewhat like the “false fold” that remains even when attempting to change the shape of the fold. Taking into account the attractors of the learner enables the target gesture adapted to the neuromotor constitution of said learner to be reproduced.

The invention also takes into account the feedback of the learner when performing the gesture. In other words, the corrective instructions of the gesture given to the learner take into account the perception of the gesture by the learner.

Hereafter, the term “learning session” denotes the period during which the method is carried out by the learner.

The method according to the invention is based on modeling the skeleton of the human body as a plurality of members interconnected by links according to a parent-child inheritance relationship so that the movement of a parent member causes the movement of each child member connected to the parent member by a link, each link being associated with a range of motion and at least one degree of freedom in space. Thus, the arrangement of sensors on at least part of the members of the learner that are found in the modeling of the human skeleton allows information to be retrieved both on the movements of the members in question and on the members linked (directly or indirectly) to these members by said inheritance relationship of the modeled skeleton.

By way of an example, a sensor placed on the pelvis allows the movements of the right and left lower members to be followed by calculating the points of articulation.

The skeleton modeling also can be adapted to the learner. To this end, a model skeleton of the learner is created on the basis of a general skeleton model and anthropometric data of the learner. This modeling of the skeleton of the learner allows a clinical past of the learner to be taken into account by characterizing the links between members in particular. Thus, it will not be possible to define a correction instruction that conflicts with anything that is anthropometrically impossible for the learner. By way of an example, a learner cannot be asked to bend the knee significantly if their clinical history prevents this movement.

Throughout the text, the term “member” denotes a part of the skeleton of a human that forms a functional whole from a biomechanical point of view and that is not necessarily limited to a single bone of the skeleton. In particular, the torso, the head or the pelvis of the skeleton each form a member within the meaning of the invention even though they are each made up of a plurality of bones and a plurality of joints.

The connections between the members obey the laws that govern conventional mechanics. Each link is associated with a range and with one or more degrees of freedom in space. For example, the elbow has only one degree of freedom, whereas the ankle has three.

The invention is designed to track the movement of the members of the skeleton using a plurality of sensors worn by the learner, thereby enabling biomechanical data to be acquired that may assume the form of quaternions, Euler angles and/or gravity vectors, for example.

The number of motion sensors and their position on the learner depends on the gesture to be learned and results from a biomechanical analysis of the movement. The method can thus comprise a step of selecting the gesture from a previously formed database that associates the number of sensors and their position on the learner with each type of gesture.

The invention is designed to analyze the acquired biomechanical data and to determine a theoretical correction of the gesture by comparing these acquired biomechanical data with biomechanical data corresponding to an expected nominal target gesture. For example, in the case of a throwing gesture, this step can compare the range of the rotation of the shoulders, measured in a transverse plane, with the nominal range.

The method is then designed to refine the proposed correction by taking into account the specific features of the learner on the basis of adaptive parameters.

These specific features can be of any type. For example, in the case of the aforementioned rotation of the shoulders, if the athlete has had spinal surgery, the target range is limited compared to the ideal range.

According to an alternative embodiment of the invention, the specific correction instruction is transmitted to the learner only after the specific correction instruction has been updated after receiving the feedback from the learner. In other words, according to this embodiment, the step of transmitting the correction instruction to the learner is subsequent to the step of updating the specific correction instruction of the learner.

According to another alternative embodiment, the transmission step is duplicated with a transmission following the customization of the specific correction instruction and another transmission following the updating of the correction instruction following the reception of the feedback from the learner.

According to an advantageous alternative embodiment of the invention, at least one predetermined adaptive parameter is derived from behavior models of the learner on the basis of a history of biomechanical data acquired for the learner.

This advantageous alternative embodiment therefore allows the adaptive parameters to be calculated on the basis of biomechanical data acquired during previous implementations of the method by the learner.

According to an advantageous alternative embodiment of the invention, at least one predetermined adaptive parameter is a biomechanical, physiological or neuromotor parameter of the learner.

A biomechanical parameter aims to take into account physical differences between the learner and a target skeleton. For example, if the learner has undergone arthrodesis of the L3 to L5 vertebrae, their range of rotation of the pelvis is limited and the biomechanical parameter allows this specific feature to be taken into account.

A physiological parameter aims to take into account a physiological difference with respect to a norm. For example, if a learner has a very low heart rate (for example, of approximately 30 beats per minute at rest), the adaptive parameter allows this specific feature to be taken into account and allows the correction instruction to be modulated accordingly. Such a physiological parameter can be provided by a dedicated sensor worn by the learner.

A neuromotor parameter aims to take into account kinesiophobic-type apprehension of a learner when faced with certain movements. For example, a learner who has just had a knee arthroscopy may be apprehensive about bending it beyond a certain limit.

The adaptive parameters can be provided before the method is carried out by the learner or can depend on the method as such. Thus, according to an advantageous alternative embodiment, the neuromotor parameter depends on information provided by the learner representing the sensation perceived when performing the corrected gesture. For example, the learner can advise that they feel tired so that a corresponding adaptive parameter modulates the correction instruction in order to take into account this feeling of fatigue.

The adaptive parameters that influence the customization of the theoretical correction to a specific correction also can be parameters representing environmental conditions. For example, it can involve the ambient temperature, the atmospheric pressure, the degree of humidity, the wind, the quality of the surface on which the gesture is performed (athletics track becoming slippery or type of snow, etc.) or the physiology of an animal directly influencing the execution of the gesture (e.g., the degree of stress of the horse ridden by the learner rider), etc.

Determining the correction instruction is based on a previously formed knowledge base.

The invention then allows the specific correction instruction to be updated on the basis of information representing the sensation perceived by the learner when performing the gesture.

This step of the invention allows the system to correct the instruction as a function of the bias perceived by the learner when performing their gesture. Indeed, a gesture is not a mechanical process which is preprogrammed in the brain, then initiated thereby and carried out by the musculoskeletal system, but results from a continuum of adaptations depending on the internal and external information received by the central nervous system of the learner. These adaptations mainly result from the sensory perception, in particular proprioceptive, that the learner has of the position of their body. This perception does not create a systematic bias, which could be detected and taken into account by the motion sensors, but evolves under the effect of stress, fatigue or optical illusions, etc. It is an original cerebral production that is a function of past, often unconscious, experiences of the physical state of the moment or ambient cognitive biases, etc.

Thus, the difference between the performed gesture and the gesture that the learner thinks of performing is constantly changing and must be taken into account in order for the correction of the gesture to be effective. Without this feedback information, the correction is monotonous and ultimately creates, through cognitive dissonance, a deleterious bias for the gesture, a form of a vicious circle which keeps a gesture from diverging from the target gesture.

The invention therefore allows the cognitive biases inherent in the activity of the central nervous system to be rectified. Indeed, the correction instruction is not directly transmitted to the musculoskeletal system, but passes through the central nervous system of the learner.

The invention therefore allows an objective improvement to be obtained in the gesture being addressed, irrespective of the mental states of the learner.

The invention can be used for any type of sport, in particular for sports that require the visual concentration of the learner when performing the gesture (such as tennis, skiing or horse riding, etc.). In particular, the correction instruction can be transmitted and this correction instruction can be taken into account by the learner without requiring the gaze of the learner so that they can fully concentrate on their gesture, while receiving the instruction and while providing their feedback.

Advantageously and according to the invention, said step of analyzing and determining a theoretical correction instruction comprises the following sub-steps:
analyzing said acquired biomechanical data in order to allow angles and projections of the points associated with said sensors to be defined on the basis of said modeled skeleton; and
comparing said defined angles and projections with angles and projections of the target gesture in order to provide a theoretical correction instruction.

According to this alternative embodiment, the biomechanical data which are acquired by the sensors worn by the learner and associated with the features of the members and the links of the skeleton model allow angles and point projections to be defined on the various biomechanical planes and allow the evolution of these various variables to be monitored during the learning session.

These variables are compared with variables of the predetermined target gesture. The predetermined target gesture can result either from the upstream acquisition of biomechanical data from an expert of the gesture to be learned, or from a simulation of the gesture.

Advantageously and according to the invention, said biomechanical data analyzed by said step of analyzing and determining a theoretical correction instruction are the data saved in a circular buffer memory enhanced with the biomechanical data acquired from the detection of a trigger signal.

This advantageous alternative embodiment allows the amount of analyzed data to be limited to only relevant data which take into account the movement to be learned. For example, in the case in which the learner only seeks to optimize hitting a tennis ball, only the data relating to the impact (or to the vicinity of the impact) may be of interest to them, in which case, the trigger signal can be provided by an impact sensor that triggers the acquisition only from the moment the ball comes into contact with the racket.

According to this advantageous alternative embodiment, the data are acquired by the motion sensors and saved in a first circular buffer memory, better known as a ring buffer. This first ring buffer has a predetermined limited size denoted by T1, corresponding to 100 storage rows, for example. When this first ring buffer is filled, the next acquisition (row 101) takes the place of the first row of the ring buffer so that this first ring buffer always comprises 100 rows of data. When the trigger signal is detected, the data are saved in a second ring buffer of size T2. The analysis of the movement then relates to the data contained in the ring buffers T1 and T2 with, if necessary, the temporal information allowing the trigger signal to be located.

This alternative embodiment is particularly advantageous for analyzing movements that have a significant latency time between two consecutive gestures, such as a tennis serve or a golf swing, for example.

Advantageously and according to the invention, said trigger signal is detected by a predetermined detection sensor or by an analysis module of the gesture carried out by the learner and configured to highlight a predetermined situation.

According to this advantageous alternative embodiment, the trigger signal is directly derived from the analysis of the movement performed by the learner. For example, in the case of hitting a tennis ball, the trajectory of the arm can be analyzed and the trigger signal corresponds to the moment when the arm accelerates in the sagittal plane. According to this alternative embodiment, the event that triggers saving data in the circular memory (which form the biomechanical data analyzed in order to determine the theoretical correction instruction) is dependent on the sport or the gesture being learned. For example, for alpine skiing, the trigger event can be the detection of a ski parallel to the slope line. This event is detected by crossing gravitation and orientation data from inertial sensors. The method according to the invention preferably comprises a database that stores, for each sport and each associated gesture which can be learned using the invention, the characterization of the trigger event.

Advantageously and according to the invention, said step of transmitting a specific correction instruction to the learner involves transmitting voice messages representing the correction to be performed by the learner.

This specific correction instruction can be transmitted using all types of means. Preferably, this transmission occurs using audio means.

The correction instructions are transcribed into one or more prerecorded keywords in a library of predetermined keywords.

This alternative embodiment is particularly advantageous insofar as it allows a correction instruction to be transmitted to the learner without distracting them from the gesture being performed. In particular, the learner does not need to divert their gaze toward a display screen or any equivalent means in order to become aware of the instruction to be applied insofar as the instruction is intended for a sense of the learner that is not very critical for performing the gesture, in this case hearing. This alternative embodiment therefore is particularly suitable for sports that need the visual concentration of the learner when performing the gesture (tennis, skiing or horse riding, etc.).

Advantageously and according to the invention, said step of updating said specific correction instruction comprises a step of receiving a voice message transmitted by the learner representing the sensation felt when performing the movement and of transcribing this voice message using a voice recognition module.

This advantageous alternative embodiment therefore allows the specific feedback of the learner to be taken into account when performing the gesture. For example, the learner can provide information that characterizes the quality of the gesture that they think they have performed by classifying their gesture on a scale of 1 to 5.

Advantageously, a method according to the invention further comprises a step of transmitting a warning signal to the learner when said performed gesture deviates from the target nominal gesture by a predetermined deviation.

This alternative embodiment allows the learner to be provided with information only if the deviation between their gesture and the target gesture moves away from a predetermined deviation.

The invention also relates to a computer program product which can be downloaded from a communication network and/or is recorded on a computer-readable medium and/or can be executed by a processor, characterized in that it comprises program code instructions for carrying out the learning method according to the invention when the program is executed on a computer.

The invention also relates to a fully or partially detachable computer-readable storage medium storing a computer program comprising a set of computer-executable instructions for carrying out the learning method according to the invention.

The invention finally relates to a system for learning a gesture by a human learner, the skeleton of said human learner being modeled by a plurality of members interconnected by links according to a parent-child inheritance relationship so that the movement of a parent member causes the movement of each child member connected to the parent member by a link, each link being associated with a range of motion and at least one degree of freedom in space, said system comprising:
a plurality of motion sensors intended to equip the learner on a plurality of predetermined members according to said gesture to be learned from among those that model said human skeleton;
a module for acquiring biomechanical data provided by said plurality of sensors during a gesture performed by the learner;
a module for analyzing said acquired biomechanical data and for comparing these biomechanical data with those corresponding to a target gesture;
a module for calculating a theoretical correction instruction for the gesture on the basis of said modeled human skeleton and of said comparison between the biomechanical data of the learner and those of the target gesture;
a module for customizing the theoretical correction instruction into a specific correction instruction on the basis of predetermined adaptive parameters related to the learner and/or to the environment;
a module for transmitting said specific correction instruction to the learner;
a module for updating said specific correction instruction on the basis of information representing the sensation perceived by the learner when performing the corrected gesture.

A system according to the invention advantageously carries out a method according to the invention and a method according to the invention is advantageously carried out by a system according to the invention.

Throughout the text, the term “module” denotes a software component, i.e., a subset of a software program, which can be compiled separately, either for independent use or to be assembled with other modules of a program, or a hardware component, or a combination of a hardware component and a software subprogram. A hardware component can comprise an integrated circuit specific to an application (better known by the acronym ASIC for Application-Specific Integrated Circuit) or a programmable logic circuit (better known by the acronym FPGA for Field-Programmable Gate Array) or a circuit of specialized microprocessors (better known by the acronym DSP for Digital Signal Processor) or any equivalent hardware or any combination of the aforementioned hardware. In general, a module is therefore a (software and/or hardware) component that enables a function to be provided.

The technical advantages and effects of the method according to the invention apply, mutatis mutandis, to a system according to the invention.

Advantageously, a system according to the invention further comprises:
sensors for measuring pressure variations on any point of support;
and/or sensors for measuring biological parameters of the learner.

These sensors, which are provided in addition to the motion sensors, are also configured to equip the learner before the learner performs the gesture.

Advantageously and according to the invention, the system comprises a voice recognition module connected to a microphone and configured to be able to interpret keywords spoken by said learner into said microphone.

Advantageously and according to the invention, the system comprises earphones intended to be worn by said learner in order to receive the gesture correction instructions.

The invention also relates to a learning method and a learning system, which are characterized in combination by all or some of the features mentioned above or hereafter.

### LIST OF FIGURES

Further aims, features and advantages of the invention will become apparent upon reading the following description, which is provided solely by way of a non-limiting example, and which refers to the accompanying figures, in which:

FIG. 1 is a schematic view of a system according to an embodiment of the invention;

FIG. 2 is a schematic view of a method according to an embodiment of the invention;

FIG. 3 is a schematic view of the variation in the speed of a tennis racket as a function of time, allowing a region of impact corresponding to a target gesture to be determined;

FIG. 4 is a schematic view of part of a modeled skeleton of a learner learning to kick a football;

FIG. 5 is a schematic view of a model of a skeleton of a learner implemented in a method according to an embodiment of the invention.

### DETAILED DESCRIPTION OF ONE EMBODIMENT OF THE INVENTION

For the sake of illustration and clarity, the figures do not strictly adhere to scales and proportions.

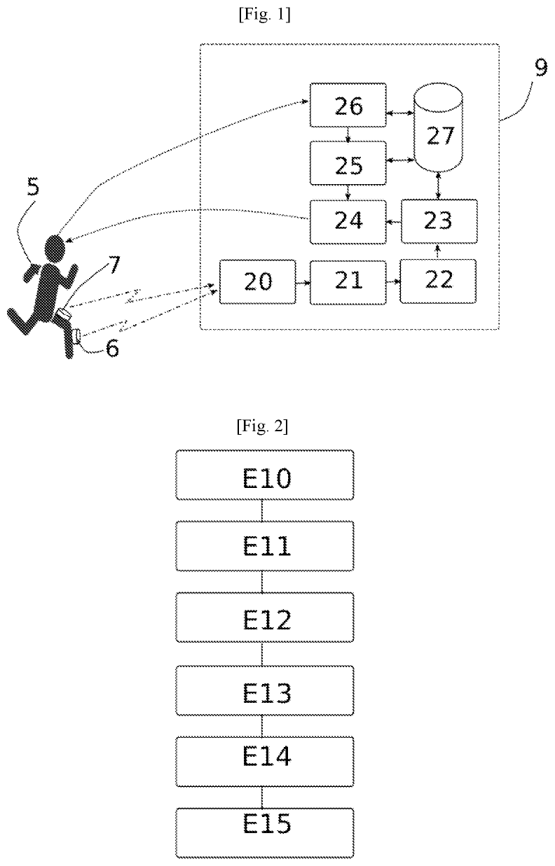

FIG. 1 schematically illustrates a learning system according to an embodiment of the invention carrying out a learning method according to an embodiment of the invention and schematically shown in FIG. 2.

The first step involves selecting the gesture being addressed. The gesture is selected from a database which is pre-established and prerecorded in the system. The selection of the gesture determines the members (also denoted using the term “bones” throughout the text) affected by the exercise in a pre-established structured set of the modeled human skeleton.

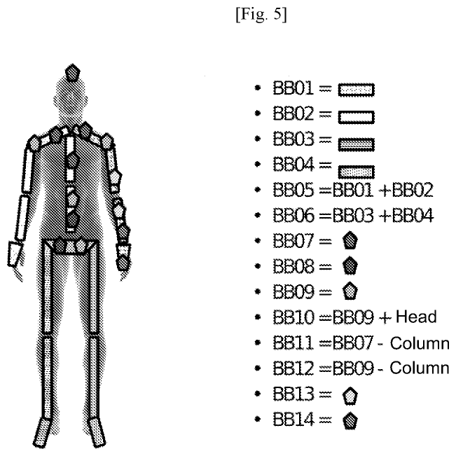

FIG. 5 schematically illustrates such a model of the human skeleton by a plurality of members linked together by links according to a parent-child inheritance relationship such that the movement of a parent member causes the movement of each child member connected to the parent member by a link, each link being associated with a range of motion and at least one degree of freedom in space.

Thus, in FIG. 5, the members are grouped into different blocks referenced BB01 for the left arm, BB02 for the right arm, BB03 for the right leg, and BB04 for the left leg. From these blocks, linked blocks can be obtained, such as block BB05, which is the sum of block BB01 and block BB02, and so on for all blocks BB06 to BB12 in FIG. 5.

Another simplified example is shown in FIG. 4 for learning to kick a football and is described hereafter.

Once the gesture and the associated members have been selected, the learner 5 is equipped with a plurality of motion sensors 6, 7. The choice of the positioning of the motion sensors on the learner depends on the movement to be learned. By way of an illustration only, the learner 5 in FIG. 1 is a runner seeking to improve their stride. Of course, the invention is not limited to this single gesture and any type of gesture can be learned from a system according to the invention.

In general, the number of motion sensors and their position on the learner depends on the gesture to be learned and results from a biomechanical analysis of the movement. The system according to the invention can thus comprise a database, not shown in the figures, which associates the number of sensors and their position on the learner with each type of gesture. Thus, the learner can select the type of gesture to be performed and can equip themselves with the corresponding sensors mentioned in the database. This database can be provided by a biomechanics expert or can be formed by a preliminary analysis of the movement.

According to an embodiment, each gesture to be learned can also be characterized by a plurality of criteria.

The first criterion, called a “sequential” criterion, aims to define whether the gesture is to be analyzed continuously or only over a portion of the gesture. By way of an example, if seeking to analyze the gesture of hitting a tennis ball, only the biomechanical data acquired in the vicinity of this hit, which is considered to be the key moment of the gesture, needs to be analyzed. In other words, the analysis of such a gesture is sequential. However, other gestures need to be analyzed continuously.

The second criterion, called a “trigger” criterion, aims to define the trigger for the analysis. With further reference to the previous example, the key moment is when the learner hits the ball. This hitting of the ball therefore needs to be detected, either from a dedicated sensor, or from an analysis of the gesture. The sensor is configured to transmit a trigger signal that is recovered by the system. It is also possible to detect this trigger signal on the basis of the analysis of the movement of the arm involved in hitting the ball. For example, the trajectory of the hitting arm is analyzed and, by construction, it is determined that, in this trajectory, the key moment is formed by the start of an acceleration in the sagittal plane of the hitting arm. It is then possible to determine the key moment of the movement to be analyzed without using a dedicated sensor.

The third criterion, called a “warning” criterion, aims to determine whether a signal is sent to the learner when the performed gesture deviates from a predetermined deviation from the target gesture.

In particular, the aim of the invention is to allow the learner to perform a gesture that is as close as possible to the target gesture. During the learning session, the learner attempts, through repetitions, to match their gesture to the target gesture. In order to progress without error, the learner needs to be informed, other than by their own sensations, of the difference between the targeted gesture and the performed gesture. With further reference to hitting a ball, it is generally accepted that the ball is ideally hit when the racket reaches its maximum speed. The juxtaposition of the impact and the acceleration curve of the racket on the same time scale means it is possible to determine whether or not the impact was optimal. A time range during which the impact is considered acceptable is therefore determined. If the impact occurs outside this time range, the system provides the learner with the “warning” signal. This signal contains additional information in relation to the gesture corrections provided by the system and described hereafter.

Once the gesture has been selected, associated if necessary with the criteria listed above, the acquisition of the data of the gesture and the processing of these data can begin.

The motion sensors 5, 6 record biomechanical data transmitted to a data acquisition module 20, for example, by wireless means.

The acquisition module 20 is implemented, for example, using software means and provides the analysis module 21 with the measurements provided by the sensors. This acquisition module 20 implements step E11 of the learning method.

The analysis module 21 extracts variables from the received measurements that allow the gesture performed by the learner to be characterized. These variables are, for example, angles, projections, supports, speed or acceleration, etc., taken in the three planes of the space.

The system can also comprise other sensors, such as biological sensors (the temperature of the learner or electrocardiogram, etc.) that enhance the analysis module 21.

The system verifies that the data received from the sensors installed on the learner are those required for the evaluation of the exercise.

Subsequently, the system reads the features of the relevant joints (degrees of freedom or relevant range of bending extension) from a prerecorded database. This database can be general (a general anthropometric database) or more specific to the learner by virtue of a clinical examination and/or the recording of previous movements of the same type.

Thereafter, the system gathers the data from the sensors for a home position assumed by the learner (calibration position).

Then, the system systematically gathers, at a frequency that is determined depending on the selected exercise, the data from the sensors placed on the corresponding members throughout the exercise that is performed.

Optionally it is possible, instead of analyzing the movements one at a time, to compute the minimum and maximum average values of a series of movements of the same type.

The analysis module 21 compares the extracted variables with corresponding variables resulting from a target gesture.

The analysis and comparison module 21 carries out the analysis and comparison step E12 of the method according to the invention.

To this end, the system reads the target values of the relevant movement from a prerecorded database formed by a specialist (biomechanic, trainer, surgeon or physiotherapist, etc.) or by the learner if they have sufficient skills. This database reflects the generally accepted learning curve of the corresponding movement.

Then, throughout the execution of the movement, the system compares the values obtained from the sensors with the target values.

Subsequently, throughout the execution of the movement the system compares the differences that are obtained with faulty movement patterns read in the “exercise” database and prerecorded by a specialist (biomechanic, trainer, surgeon or physiotherapist, etc.) or by the learner if they have sufficient skills.

The system also comprises a module 22 for calculating a theoretical correction instruction configured to supply a theoretical correction instruction for the gesture performed by the learner on the basis of the analysis and of the comparison carried out by the module 21.

To this end, the system identifies a relevant pattern by comparing the pattern with the data of the performed movement (with simple or more complex algorithms, such as Bayesian inferences, for example).

Then, the system reads, from a database prerecorded by a specialist (biomechanic, trainer, surgeon or physiotherapist, etc.) or by the learner if they have sufficient skills, the contents of the instructions corresponding to the identified faulty pattern.

Subsequently, the system reads, from a database prerecorded by a specialist (biomechanic, trainer, surgeon or physiotherapist, etc.) or by the learner if they have sufficient skills, the contents of the constraints for transmitting the instructions corresponding to the identified pattern (latency time between the end of the movement and the transmission of the instruction, for example).

The system also comprises a module 23 for customizing the theoretical correction to a specific correction.

This customization is based on parameters stored in a database 27 and/or on the result of an analysis of previous movements performed by the learner.

To this end, the system analyzes the movements recorded since the start of the exercise (moving averages, standard deviations, minima or maxima, etc.).

Then, the system compares the results of the analysis with the target values and modifies the learning curve if the observed differences are notably different from the basic learning curve. The definition of the notable difference can be set and can correspond to a distance measurement according to a predetermined metric of the measured values with respect to the target values.

Then, and as is the case for determining the theoretical correction instruction, the system identifies a relevant pattern by comparing the pattern with the data of the performed movement (with simple or more complex algorithms, such as Bayesian inferences, for example).

Subsequently, the system reads, from a database prerecorded by a specialist (biomechanic, trainer, surgeon or physiotherapist, etc.), the contents of the instructions corresponding to the identified faulty pattern.

The customization module 23 carries out the customization step E13 of a method according to the invention.

The system also comprises a module 24 for transmitting a personalized instruction to the learner 5. This module is preferably associated with audio means configured to transmit the instructions to the learner.

This transmission module 24 carries out the transmission step E14 of the method according to the invention.

Finally, the learner 5 can provide the system with feedback, which is then analyzed by the voice recognition module 26, which relies on the database 27 to interpret the transmitted feedback.

This allows the updating module 25 to update the correction instruction and to re-transmit it to the learner via the transmission module 24.

To this end, the system gathers the reactions of the learner transmitted by the microphone in the form of keywords previously recorded in a database and known to the learner, depending on the exercise in question. Only contextually relevant keywords are retained by the speech recognition feature.

Then, the system identifies the modification that these keywords can apply to the current instruction from the same database.

The recognition module 26 and updating module 25 carry out the step E15 of updating the method according to the invention.

The method will now be described using the example of a tennis forehand that the learner is seeking to improve.

The development of the theoretical correction targeted by step E12 is based, for example, on the variation of the speed of the racket over time, with it being understood that the aim is to hit the ball when the racket reaches a maximum speed.

The motion sensor used is, for example, an inertial unit placed on the back of the hand of the learner holding the racket. This inertial unit transmits quaternions and the raw values of the accelerometer of the unit to the computing unit at a frequency of 50 Hz, for example.

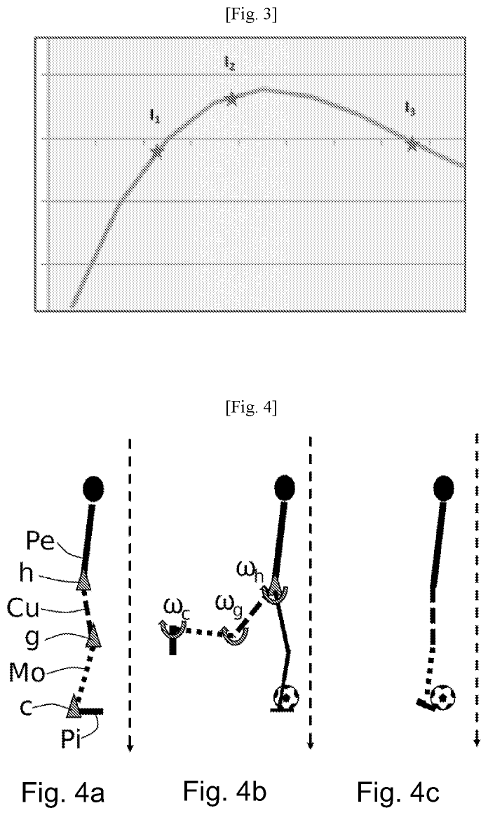

The data provided by the inertial unit allows the curve which is schematically shown in FIG. 3 to be obtained.

The motion sensor also detects the hitting moment and determines whether this hit occurred within the time window corresponding to the maximum hitting speed. As shown in FIG. 3, the curve representing the variation in the speed of the racket as a function of time is split into three distinct zones which correspond to a hit before the maximum speed, a hit at the maximum speed, and a hit after the maximum speed, respectively. Thus, the impact I1 occurs before the maximum speed zone. The impact 13 occurs after the maximum speed zone. The impact I2 occurs in the maximum speed zone.

Thus, the theoretical correction can involve developing an instruction of the “hit too early” type for impact I1, “ideal hit” for impact I2 and “hit too late” for impact 13.

In step E13, the theoretical correction instruction is customized by taking into account the attractors of the learner. To this end, the database 27 is polled. By way of an example, the database 27 reveals that the learner has the specific feature of stiffening their wrist when they hit the ball in the maximum speed zone, which results in an inaccurate hit.

In order to obtain the target gesture, in the case of this learner, it is therefore necessary to rectify the optimal hitting zone by slightly shifting it earlier in the time scale (lower speed) in order to avoid this inaccuracy. Once this correction has been made, the effective correction instruction would become the following, for example: for I₁: “ideal hit,” for I₂: “hit too late” and for I₃ “hit too late.”

It is also possible to take into account adaptive parameters linked to the environment, for example, information representing the surface of the tennis court (which becomes slippery due to a rain shower and which therefore modifies the rebound of the ball and the supports). This environment parameter then involves increasing the tolerance for error in the gesture and therefore expanding the zone of maximum acceptable speed used to establish the correction.

In step E14, the specific correction instruction thus established is transmitted to the learner.

In step E15, the learner provides the system with their feedback when hitting the ball. The learner has, for example, a determined period after the impact to transmit their feedback of the impact. This is transmitted via a microphone using the following keywords: “early”; “correct”; “late.”

The matrix of the following final correction messages is then obtained:

TABLE

Impact

| Feedback | I₁ | I₂ | I₃ |
|---|---|---|---|
| “early” | No, correct | No, late | No, late |
| “correct” | OK | No, late | No, late |
| “late” | No, correct | OK, late | OK, late |

The updated correction instruction in the matrix above is then transmitted to the learner.

According to one embodiment, there is a provision that involves not transmitting any correction instruction before having received the feedback from the learner. In other words, the transmission step E14 is only carried out after the step E15 of receiving the feedback.

According to another embodiment, there is a provision that involves transmitting the specific correction instruction that takes into account the attractors alone, then receiving the feedback information and establishing a second correction, as described above.

According to other embodiments, a provision also can be made that involves transmitting the correction instruction only after a certain time or a certain number of gestures. A provision also can be made that involves transmitting the messages only if the gestures are persistently inadequate.

In this case, a parameter will be available in the database 27 that relates to the minimum duration of the fault or the number of faulty movements (a chest bent too far for too long when horse riding or skiing, for example). This thus avoids “false positives,” i.e., a leaning chest for a fraction of a second after traveling over uneven ground.

In the same spirit as above, physiological parameters (fatigue, for example) can be taken into account.

In such a case, the attractor taken into account in step E13 can involve specifying that the stiffening of the wrist only occurs from a certain level of fatigue. Thus, the variation in heart rate, associated with the raw frequency, can be used as an indicator of fatigue. This heart rate is acquired by a heart rate monitor worn by the learner.

Another embodiment of the invention is described in relation to FIG. 4 and kicking a football.

FIG. 4 schematically illustrates the members and links between members carried out within the context of kicking a football. FIG. 4a illustrates the position of the members at rest, FIG. 4b illustrates the position of the members during the run-up and FIG. 4c illustrates the position of the members when the foot hits the ball.

The skeleton modeling shows the following parent-child biomechanical set:
Pelvis member (reference Pe in FIG. 4): Hip joint (3 degrees of freedom, reference h in FIG. 4);
Thigh member (reference Cu in FIG. 4): Knee joint (1 degree of freedom, reference g in FIG. 4);
Calf member (reference Mo in FIG. 4): Ankle joint (3 degrees of freedom, reference c in FIG. 4);
Foot member (reference Pi in FIG. 4).

Furthermore, the variable ωₕ denotes the angular speed of the pelvis-thigh joint in the sagittal plane. The variable ω_{g} denotes the angular speed of the thigh-calf joint in the sagittal plane. Finally, the variable ω_{c} denotes the angular speed of the calf-foot joint in the sagittal plane.

The sub-set of the modeled skeleton is therefore made up of four members (pelvis, thigh, calf and foot) and three joints (hip, knee, ankle), the parent-child relationship of which can be represented as follows:

PELVIS [symbol] hip [symbol] THIGH [symbol] knee [symbol] CALF [symbol] ankle [symbol] FOOT

The members are considered to be rigid solids with dimensions that are provided by anthropometric tables, for example.

Learning to hit a ball according to the method of the invention involves the following.

1. Installation of Sensors

The first step involves equipping the four relevant members with an inertial measurement unit sensor (better known by the acronym IMU) in order to be able to provide rotation values for each member, for example, the three Euler angles x, y, z or the quaternions x, y, z, w.

In this case, the axis providing the rotation values of the axes of the sagittal plane is kept close to the gravity vector (represented by the dashed downward arrow).

Since they are rigid solids with a known length, knowing the rotations allows the corresponding relative translation movements to be known (i.e., the movements of the segments).

2. Biomechanical Data Analysis

The biomechanical analysis of the data provided by the sensors involves the following.

2.1. Identification of the Values in the Rest Phase.

Firstly, the values in the rest phase are identified. To this end, the values of the angles in the rest position are recorded and stored. Their angulation with respect to the gravity vector is also recorded.

2.2. Identification of the Mobilizing Phase and the Initiating Phase.

Secondly, the mobilizing phase and the initiating phase are identified.

The initiating phase begins when the thigh-calf-foot segments begin their forward movement of the body.

The mobilizing phase and the initiating phase therefore can be identified by analyzing the direction of the variation of the angular speeds on the time scale. Thus, for each predetermined time interval:
if ω_{g}-ω_{g-1} is negative, then it is the mobilizing phase;
if ω_{g}-ω_{g-1} is positive, then it is the initiating phase.

The value of the angle ωₑ of each joint at the end of the run-up and at each instant to be analyzed, in particular during impact, is also recorded.

2.3. Identification of the Impact

The impact is identified on the time chain by analyzing the raw acceleration signal from the sensor placed on the foot of the player. The impact on the ball actually generates a characteristic imprint on the signal from this sensor.

2.4. Analysis of the Obtained Values

The various values are compared with the desired angulations (at the end of the run-up, halfway and during impact, etc.).

The objective in this case is to maximize the sum of the angular speeds of the segments so that the value of the force delivered on impact by the studied segment (the foot) is the maximum force.

3. Determination of the Theoretical Correction Instruction

General instructions can be determined on the basis of the obtained values, for example, by comparing the angular values at the end of the run-up phase with the values generally obtained by comparable learners of comparable morphology and age. These values are read from a database. If ω_{achieved}<ω_{objective}, then the theoretical instruction is “mobilize more.”

4. Determination of the Specific Correction Instruction for the Learner

Replacing the general data with data specific to the learner makes it possible to note, for example, that said learner has a medical history on one of the relevant joints, namely the knee. The database provides the maximum angulation values specific to this learner following their operation.

It then can be seen that ω_{achieved} is no longer less than ω_{rectified objective}.

Consequently, the condition: if ω_{achieved}<ω_{rectified objective} is no longer met.

The general instruction “mobilize more,” which appears to be unsuitable even though the range generated on initiation is correct, is therefore no longer issued.

5. Updating the Specific Correction Instruction According to the Feedback from the Learner

In order to illustrate this step, the learner is assumed to be feeling pain in the knee when hitting the ball. They send this information to the system, for example, by pronouncing the keywords “knee pain,” from among a plurality of feedback keywords prerecorded in the system and associated with the gesture being learned.

Using the example of the case whereby the system determines, by consulting a database that is pre-established and an integral part of the system according to the invention, the following information:
the learner, operated on the knee, suffers from kinesiophobia when the knee is bent more than 40° when mobilizing;
the clinical examinations allow it to bend 60° during the mobilizing phase;
the bending observed during the mobilizing movement in question, ω_{achieved}, is 45°.

The system will therefore issue a corrected instruction of the following type: “Kinesiophobia! Mobilize more!” in order to counter this kinesiophobia while respecting the clinical constraints.

Obviously, the example provided in relation to kicking a football and with the clinical history of the learner is only an example, and a person skilled in the art easily understands that the invention can be applied to any type of movement and can take into account any information related to the learner. To this end, the system clearly needs to be provided with the necessary information and the various databases polled during the method according to the invention need to be formed.

The various modules of the system according to the embodiment of the figures can be integrated into computer equipment 9 comprising a processor, a storage memory and means for communicating with the motion sensors and the means for interactively exchanging with the learner.

The various modules of a system according to the invention and the associated database can, according to one embodiment of the invention, be remote on a remote cloud server or any equivalent means. In this remote embodiment, the data provided by the motion sensors and the other sensors of the system are transmitted to the modules of the system by communication means of all types, such as wired networks or wireless networks, for example. A wired network equally can be an electrical network, an optical network, a magnetic network and in general any type of network allowing data to be transmitted. A wireless network can be of any known type, secure or unsecure. Such a network is, for example, a Wi-Fi network (i.e. according to the IEEE 802.11 standard), but it is understood that the invention applies to any wireless technology. Other radio wave technologies such as WiMax®, Bluetooth®, 3G, 4G or 5G technology particularly can be cited.

The invention is not limited to the described embodiments alone. In particular, the invention can be applied to all types of gesture and to all types of learning once the system has data representing the target gesture.

The invention also can be used to improve the cohesion between a learner and an external “system,” such as the cohesion between a horse and its rider, for example. To this end, the horse and the rider are equipped with motion sensors, with the pairing formed by the horse and the rider then forming the learner of the system according to the invention.

It is then possible to measure a certain number of identical biomechanical and physiological parameters on the rider and on the horse, to relate the results of the two measurements on a time scale, to determine the deviations and to combine them, to define a synthetic index describing the evolution of this cohesion over a time scale, and to compare this index for a given rider-horse pairing with the values obtained by experts in the technical field.

1. A method for learning a gesture by a human learner, the skeleton of said human learner being modeled by a plurality of members interconnected by links according to a parent-child inheritance relationship so that the movement of a parent member causes the movement of each child member connected to the parent member by a link, each link being associated with a range of motion and at least one degree of freedom in space, said method comprising the following steps:
equipping said learner with a plurality of motion sensors on a plurality of members that are predetermined in accordance with said gesture to be learned from among those that model said human skeleton;
acquiring biomechanical data provided by said plurality of sensors during a gesture performed by the learner;
analyzing said acquired biomechanical data and determining a theoretical correction instruction for the gesture on the basis of said modeled human skeleton and by comparing said biomechanical data of the learner with biomechanical data corresponding to a nominal target gesture;
customizing said theoretical correction instruction into a specific correction instruction on the basis of predetermined adaptive parameters related to the learner and/or to the environment;
transmitting said specific correction instruction to the learner;
updating said specific correction instruction on the basis of information representing the sensation perceived by the learner when performing the corrected gesture.

2. The learning method according to claim 1, characterized in that at least one predetermined adaptive parameter is derived from behavior models of the learner derived from a history of biomechanical data acquired for the learner.

3. The learning method according to claim 1, characterized in that at least one predetermined adaptive parameter is a biomechanical, physiological or neuromotor parameter of the learner.

4. The learning method according to any claim 1, characterized in that said step of analyzing and determining a theoretical correction instruction comprises the following sub-steps:
analyzing said acquired biomechanical data in order to allow angles and projections of the points associated with said sensors to be defined on the basis of said modeled skeleton;
comparing said defined angles and projections with angles and projections of the target nominal gesture in order to provide a theoretical correction instruction.

5. The learning method according to any claim 1, characterized in that said biomechanical data analyzed by said step of analyzing and determining a theoretical correction instruction are the data saved in a circular buffer memory enhanced with the biomechanical data acquired from the detection of a trigger signal.

6. The learning method according to claim 5, characterized in that said trigger signal is detected by a predetermined detection sensor or by a gesture analysis module produced by the learner and configured to highlight a predetermined situation.

7. The learning method according to claim 1, characterized in that said step of transmitting a specific correction instruction to the learner involves transmitting voice messages representing said specific correction instruction to be performed by the learner.

8. The learning method according to claim 1, characterized in that said step of updating said specific correction instruction comprises a step of receiving a voice message transmitted by the learner representing the sensation felt when performing the movement and of transcribing this voice message using a voice recognition module.

9. The learning method according to claim 1, characterized in that it further comprises a step of transmitting a warning signal to the learner when said performed gesture deviates from the target nominal gesture by a predetermined deviation.

10. A system for learning a gesture by a human learner, the skeleton of said human learner being modeled by a plurality of members interconnected by links according to a parent-child inheritance relationship so that the movement of a parent member causes the movement of each child member connected to the parent member by a link, each link being associated with a range of motion and at least one degree of freedom in space, said system comprising:
a plurality of motion sensors intended to equip the learner on a plurality of predetermined members that model said human skeleton in accordance with said gesture to be learned;
a module for acquiring biomechanical data provided by said plurality of sensors during a gesture performed by the learner;
a module for analyzing said acquired biomechanical data and for comparing these biomechanical data with those corresponding to a target gesture;
a module for calculating a theoretical correction instruction for the gesture on the basis of said modeled human skeleton and of said comparison between the biomechanical data of the learner and those of the target gesture;
a module for customizing the theoretical correction instruction into a specific correction instruction on the basis of predetermined adaptive parameters related to the learner and/or to the environment;
a module for transmitting a specific correction instruction to the learner;
a module for updating said specific correction instruction on the basis of information representing the sensation perceived by the learner when performing the corrected gesture.

11. The system according to claim 10, characterized in that it further comprises sensors for measuring pressure variations on any point of support.

12. The system according to claim 10, characterized in that it further comprises sensors for measuring biological parameters of the learner.

13. The system according to claim 10, characterized in that it comprises a voice recognition module connected to a microphone and configured to be able to interpret keywords spoken by said learner into said microphone.

14. The system according to claim 10, characterized in that it comprises earphones intended to be worn by said learner in order to receive said gesture correction instructions.

15. A computer program product which can be downloaded from a communication network and/or is recorded on a computer-readable medium and/or can be executed by a processor, characterized in that it comprises program code instructions for carrying out a learning method when the program is executed on a computer, the learning method for learning a gesture by a human learner, the skeleton of said human learner being modeled by a plurality of members interconnected by links according to a parent-child inheritance relationship so that the movement of a parent member causes the movement of each child member connected to the parent member by a link, each link being associated with a range of motion and at least one degree of freedom in space, said method comprising the following steps:
equipping said learner with a plurality of motion sensors on a plurality of members that are predetermined in accordance with said gesture to be learned from among those that model said human skeleton;
acquiring biomechanical data provided by said plurality of sensors during a gesture performed by the learner;
analyzing said acquired biomechanical data and determining a theoretical correction instruction for the gesture on the basis of said modeled human skeleton and by comparing said biomechanical data of the learner with biomechanical data corresponding to a nominal target gesture;
customizing said theoretical correction instruction into a specific correction instruction on the basis of predetermined adaptive parameters related to the learner and/or to the environment;
transmitting said specific correction instruction to the learner;
updating said specific correction instruction on the basis of information representing the sensation perceived by the learner when performing the corrected gesture.

16. (canceled)
